(19) United States
(12) Patent Application Publication
(10) Pub. No.: US 2024/0424204 A1
(43) Pub. Date: Dec. 26, 2024

(54) INTERLOCKED SENSOR ASSEMBLY FOR INFUSION SYSTEM

(71) Applicant: CareFusion 303, Inc., San Diego, CA (US)

(72) Inventor: Daniel Abal, San Diego, CA (US)

(21) Appl. No.: 18/693,711

(22) PCT Filed: Sep. 22, 2021
(86) PCT No.: PCT/US21/51571

§ 371 Date: Mar. 20, 2024

(51) Int. Cl. A61M 5/168; A61M 5/142

(52) U.S. Cl. CPC A61M 5/16854; A61M 5/14216; A61M 5/14228; A61M 2005/16868; A61M 2205/0216; A61M 2205/106; A61M 2205/123; A61M 2205/27; A61M 2205/3327; A61M 2205/3331

(57) ABSTRACT

A sensor assembly for an infusion system is provided. The sensor assembly includes a body, a pressure diaphragm, a fluid passageway formed between the body and pressure diaphragm, and a sensor. The pressure diaphragm includes a diaphragm interface. The sensor includes a sensor interface that is coupled to the diaphragm interface in an interlocked connection. The interlocked connection is configured to transfer a load from the pressure diaphragm to the sensor. The load may indicate a fluid pressure of the fluid passing through the fluid passageway.

### TECHNICAL FIELD

The subject matter described herein relates generally to the dispensation of fluids and more specifically to an interlocked sensor assembly for an infusion system for the delivery of fluid medications.

### BACKGROUND

Fluid pumps, such as infusion pumps, administer therapy to patients by delivering a medication or other fluid to the patient. The pumps may be connected with a fluid delivery tube, such as intravenous tubing or other administration sets. Delivery of the fluid may be adjusted based on the fluid pressure within the fluid delivery tube to help ensure that the patient is properly treated. During delivery of the fluid, movement of the patient or other sources may cause the fluid pressure within the fluid delivery tube to fluctuate, which if left unmonitored, may lead to unintended bolus, under-delivery, and/or cessation of the fluid delivered to the patient, causing various medical complications. Also, occlusions along a fluid passageway may impact the flow of the fluid to the patient. As a result, occlusions may similarly lead to unintended bolus, under-delivery, and/or cessation of the fluid delivered to the patient, causing various medical complications. Sensors and detection methods, such as direct flow-rate measurements, which are used to measure the fluid pressure, may generally be unreliable, inaccurate, and require a significant amount of device resources (e.g., power, processing time, memory, network bandwidth, and the like), especially for low flow rates of the fluid delivered to the patient.

### SUMMARY

Systems, methods, and articles of manufacture, including computer program products, are provided for using an interlocked sensor assembly with an infusion system to accurately and quickly detect a fluid pressure of the fluid passing through the infusion system and occlusions or other disturbances in the flow of the fluid.

According to some aspects, a sensor assembly for an infusion system includes a body, a pressure diaphragm coupled to the body, a fluid passageway formed between the body and the pressure diaphragm, and a sensor. The pressure diaphragm includes a diaphragm interface. The fluid passageway allows a fluid to pass therethrough to be delivered to a patient. The sensor includes a sensor interface coupled to the diaphragm interface in an interlocked connection. The interlocked connection transfers a load from the pressure diaphragm to the sensor. The load indicates a fluid pressure of the fluid passing through the fluid passageway.

In some aspects, the fluid passing through the fluid passageway is configured to cause the pressure diaphragm to move from a first position to a second position. The movement of the pressure diaphragm from the first position to the second position configured to generate the load.

In some aspects, the first position is an expanded position and the second position is a compressed position.

In some aspects, the first position is a compressed position and the second position is an expanded position.

In some aspects, the fluid pressure is a negative pressure when the pressure diaphragm is in the compressed position. The fluid pressure is a positive pressure when the pressure diaphragm is in the expanded position.

In some aspects, the pressure diaphragm includes an inner side and an outer side opposite the inner side. The outer side includes the diaphragm interface. The fluid passageway is formed between the inner side and the body.

In some aspects, the pressure diaphragm defines a flexible membrane.

In some aspects, the inner side of the pressure diaphragm surrounds an interior volume of the pressure diaphragm.

In some aspects, the pressure diaphragm includes a base portion coupled to the body, a central portion, and an interface portion includes the diaphragm interface.

In some aspects, the central portion is flexible to permit movement of the pressure diaphragm in a radially outward direction.

In some aspects, the central portion includes one or more of a fold and a curve.

In some aspects, the sensor assembly includes a body stop extending into an interior volume of the pressure diaphragm. The body stop is configured to prevent the pressure diaphragm from collapsing when the sensor assembly is coupled to a fluid administration set.

In some aspects, the fluid administration set includes one or more fluid delivery tubes.

In some aspects, the diaphragm interface includes a diaphragm mating feature, and the sensor interface includes a sensor mating feature that corresponds to the diaphragm mating feature.

In some aspects, the diaphragm mating feature includes one or more of a first shape, a first angle, and a first edge, and the sensor mating feature includes one or more of a second shape corresponding to the first shape, a second angle corresponding to the first angle, and a second edge corresponding to the first edge.

In some aspects, the sensor interface defines a protrusion, and the pressure diaphragm interface defines a receptacle configured to receive the protrusion.

In some aspects, the body forms a part of a cassette for the infusion system. The cassette includes a fluid chamber and a pumping mechanism. The fluid chamber is defined at least in part by the pressure diaphragm. The fluid chamber is configured to store an amount of the fluid. The pumping mechanism is configured to cause the fluid from within the fluid chamber to flow through the fluid passageway to be delivered to the patient. In some aspects, the pumping mechanism includes a piston.

In some aspects, the pumping mechanism is configured to operate in a first state and the pumping mechanism is configured to allow fluid to fill the fluid chamber in the first state.

In some aspects, the pumping mechanism is configured to operate in a second state, and the pumping mechanism is configured to cause the fluid to flow from the fluid chamber to the patient in the second state.

In some aspects, pumping mechanism is configured to alternate between operation in the first state and operation in the second state.

In some aspects, the body is coupled to a flow stop. Operation of the flow stop is configured to one or more of: allow the fluid to flow through the fluid passageway for delivery to a patient and prevent the fluid from flowing through the fluid passageway for delivery to the patient.

In some aspects, the body is coupled to a peristaltic mechanism. Operation of the peristaltic mechanism is configured to one or more of: allow the fluid to flow through the fluid passageway for delivery to a patient and prevent the fluid from flowing through the fluid passageway for delivery to the patient.

In some aspects, an infusion set includes the sensor assembly and an infusion device of the infusion system. In some aspects, the infusion set includes an upstream tubing configured to be coupled to the sensor assembly. The upstream tubing defines at least a part of the fluid passageway. The upstream tubing further allows the fluid to flow from a fluid storage to the sensor assembly.

In some aspects, the fluid infusion set includes a downstream tubing configured to be coupled to the sensor assembly. The downstream tubing defines at least a part of the fluid passageway. The downstream tubing is configured to allow the fluid to be delivered to a patient.

In some aspects, the infusion device is one or more of: a cassette pump, a flow stop, and a peristaltic pump.

In some aspects, the infusion device is configured to receive the sensor assembly.

In some aspects, the infusion device is configured to be coupled to the sensor assembly.

In some aspects, the fluid infusion set includes a controller. The controller includes at least one data processor and at least one memory storing instructions, which when executed by the at least one data processor, result in operations includes: causing fluid to flow through the fluid passageway to be delivered to the patient.

In some aspects, the operations further include: causing the fluid passageway to be filled with the fluid with the fluid.

In some aspects, the operations further include receiving, by the controller, the load measured by the sensor, and converting, by the controller, the load to the fluid pressure.

In some aspects, the operations further include detecting, by the controller and based on the fluid pressure, an occlusion along the fluid passageway.

In some aspects, the operations further include preventing, based on the detecting, delivery of the fluid by the infusion device.

In some aspects, the operations further include transmitting, based on the detecting, an alert.

In some aspects, the detecting includes: comparing the fluid pressure to a threshold fluid pressure, and determining the fluid pressure is less than or equal to the threshold fluid pressure.

In some aspects, the occlusion includes one or more of a blockage in the fluid passageway, a presence of air in the fluid passageway, and an obstruction in the fluid passageway.

According to some aspects, a method includes causing fluid to flow through a fluid passageway of a sensor assembly for an infusion device. The sensor assembly includes: a body, a pressure diaphragm coupled to the body, and a sensor. The pressure diaphragm includes a diaphragm interface. The sensor includes a sensor interface coupled to the diaphragm interface in an interlocked connection. The interlocked connection is configured to transfer a load from the pressure diaphragm to the sensor. The fluid passageway is formed between the body and the pressure diaphragm. The method may further include determining, based on the load, a fluid pressure of the fluid. The method may also include detecting, based on a comparison of the fluid pressure to a threshold pressure, an occlusion in the fluid passageway. The method may also include preventing, based on the detecting, delivery of the fluid to a patient.

In some aspects, the detecting further includes determining the fluid pressure is less than or equal to the threshold pressure.

In some aspects, the method also include transmitting, based on the detecting, an alert.

In some aspects, the method further includes receiving the load measured by the sensor, and converting the load to the fluid pressure.

In some aspects, the occlusion includes one or more of a blockage in the fluid passageway, a presence of air in the fluid passageway, and an obstruction in the fluid passageway.

In some aspects, the method further includes adjusting, based on the detecting, one or more parameters of the infusion device.

According to some aspects, a method of assembling an infusion set includes providing an infusion device, coupling the infusion device to a fluid administration set includes one or more fluid delivery tubes, and coupling a sensor assembly to the infusion device. In some aspects, the coupling includes snapping the sensor assembly into the infusion device.

According to some aspects, a method of assembling an infusion system includes coupling a pressure diaphragm to a body of the infusion system. The pressure diaphragm includes a diaphragm interface. The coupling forms a fluid passageway between the body and the pressure diaphragm. The fluid passageway is configured to allow a fluid to pass therethrough to be delivered to a patient. The method may also include interlocking a sensor interface of a sensor with the diaphragm interface of the pressure diaphragm. Interlocking the sensor interface with the diaphragm interface is configured to transfer a load from the pressure diaphragm to the sensor. The load indicates a fluid pressure of the fluid passing through the fluid passageway.

In some aspects, the body forms a part of an infusion device of the infusion system.

In some aspects, the method includes coupling the body to an infusion device of the infusion system.

In some aspects, the method includes replacing the pressure diaphragm with a second pressure diaphragm, and interlocking the sensor interface of the sensor with a second diaphragm interface of the second pressure diaphragm.

According to some aspects, a method of assembling an infusion set includes coupling a pressure diaphragm to a body. The pressure diaphragm includes a diaphragm interface configured to interface with a sensor interface of a sensor in an interlocked connection. The interlocked connection is configured to transfer a load from the pressure diaphragm to the sensor. The coupling forms a fluid passageway between the pressure diaphragm and the body. The load indicates a fluid pressure of fluid passing through the fluid passageway. The method may also include coupling a fluid pumping segment to a first portion of the body. The method may further include coupling a fluid delivery tube to a second portion of the body. The fluid passageway is configured allow the fluid to pass from the fluid pumping segment through the fluid passageway to the fluid delivery tube.

Implementations of the current subject matter can include methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to an infusion system having an interlocked sensor assembly, it should be readily understood that such features are not intended to be limiting. The claims that follow this disclosure are intended to define the scope of the protected subject matter.

DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

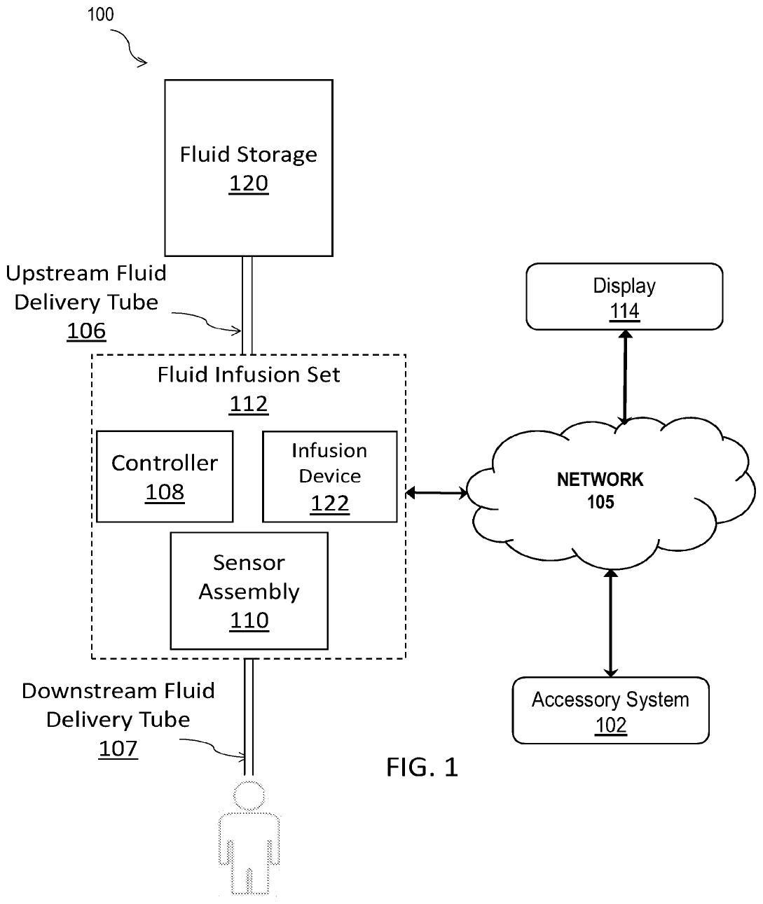

FIG. 1 depicts a system diagram illustrating an infusion system, in accordance with some example implementations;

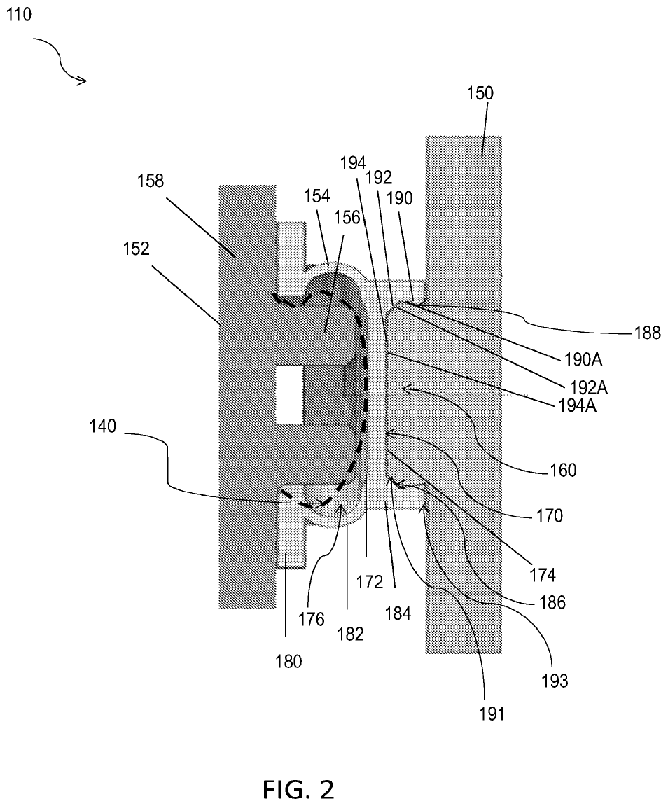

FIG. 2 schematically depicts a cross-sectional view of an example sensor assembly, in accordance with some example implementations;

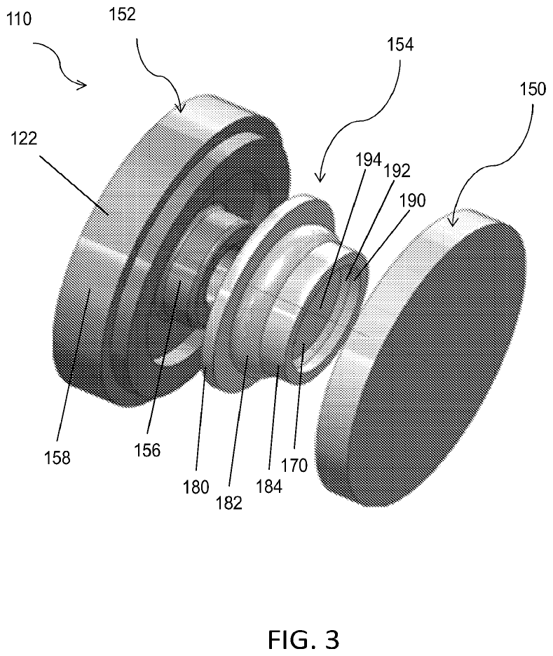

FIG. 3 depicts an exploded view of an example sensor assembly, in accordance with some example implementations;

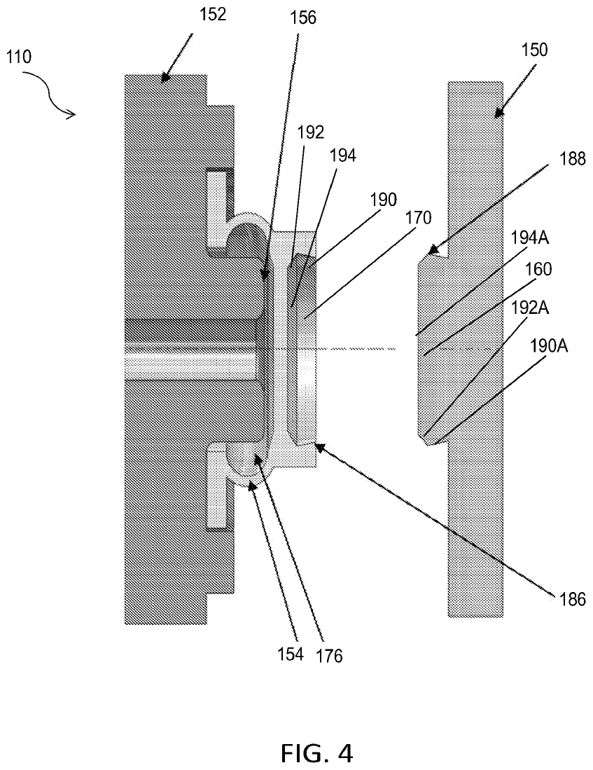

FIG. 4 depicts a cross-sectional view of an example sensor assembly, in accordance with some example implementations;

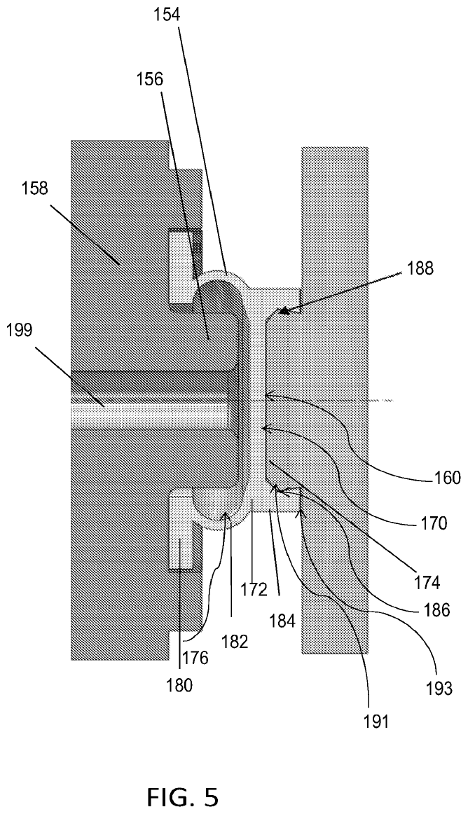

FIG. 5 depicts a cross-sectional view of an example sensor assembly, in accordance with some example implementations;

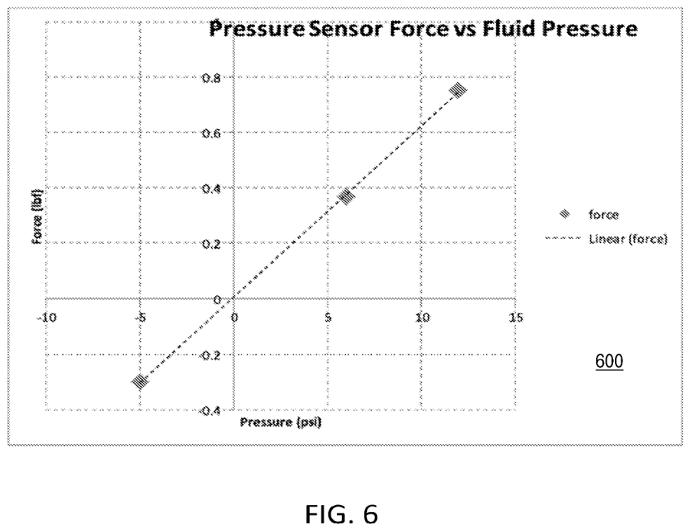

FIG. 6 depicts a graph showing a correlation between a measured load and fluid pressure, in accordance with some example implementations;

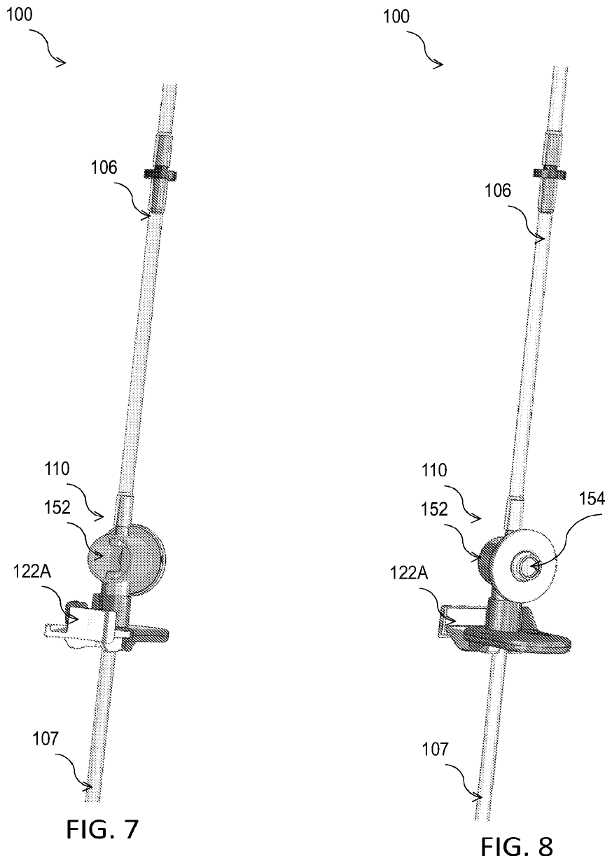

FIG. 7 depicts an example infusion system including a sensor assembly, in accordance with some example implementations;

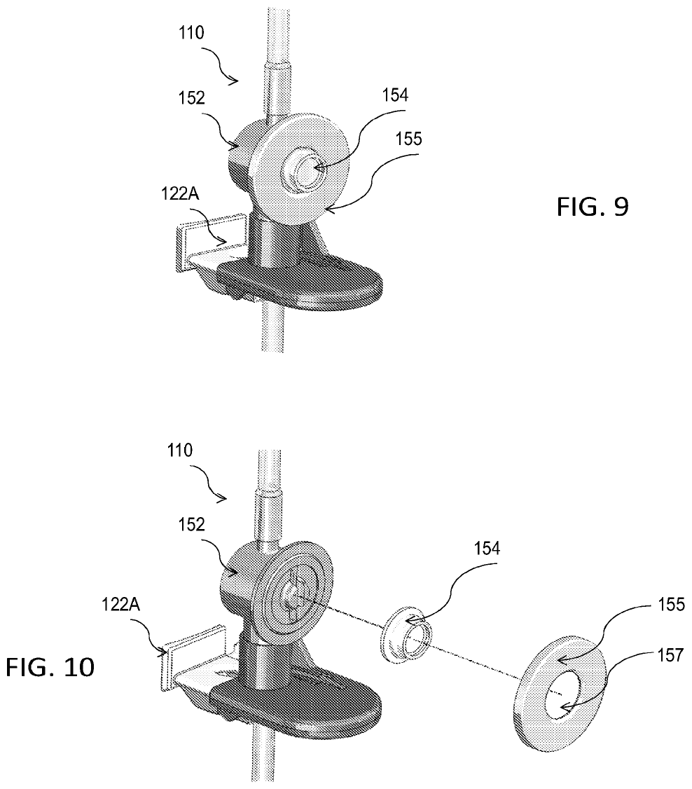

FIG. 8 depicts an example infusion system including a sensor assembly, in accordance with some example implementations;

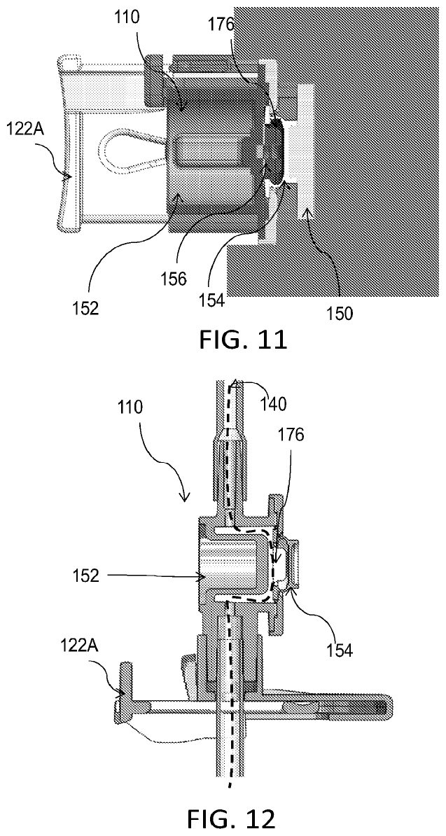

FIG. 9 depicts an example infusion system including a sensor assembly, in accordance with some example implementations;

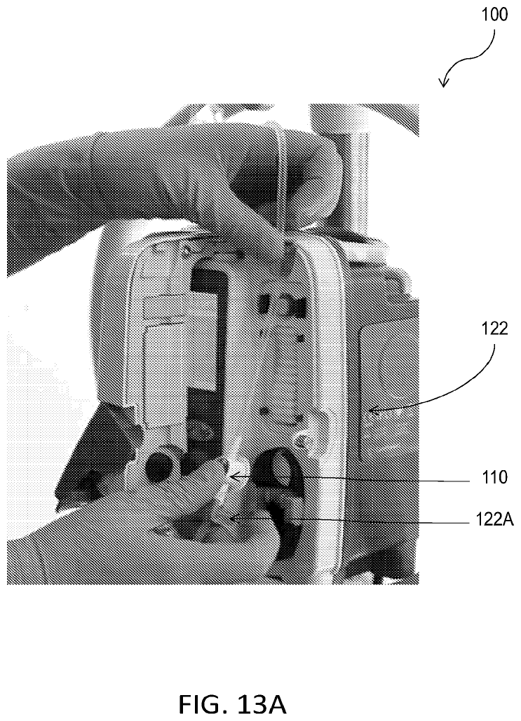

FIG. 10 depicts a partial exploded view of an example infusion system including a sensor assembly, in accordance with some example implementations;

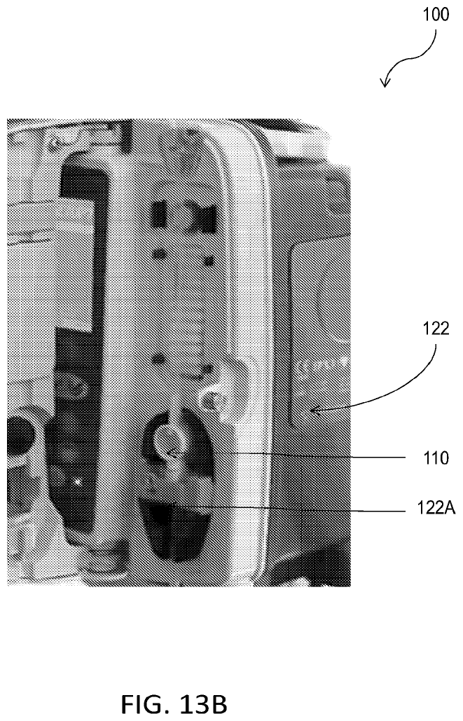

FIG. 11 depicts a top cross-sectional view of an example infusion system including a sensor assembly, in accordance with some example implementations;

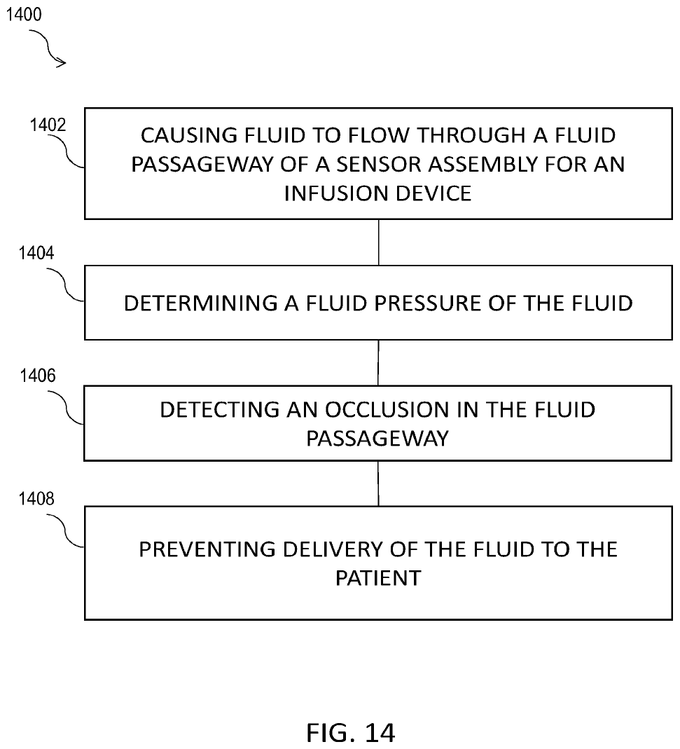

FIG. 12 depicts a side cross-sectional view of an example infusion system including a sensor assembly, in accordance with some example implementations;

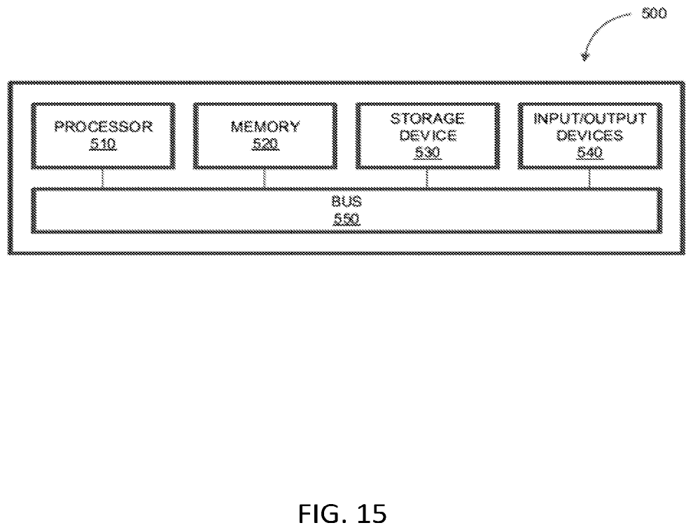

FIGS. 13A-13B depict an example sensor assembly coupled to an infusion device, in accordance with some example implementations;

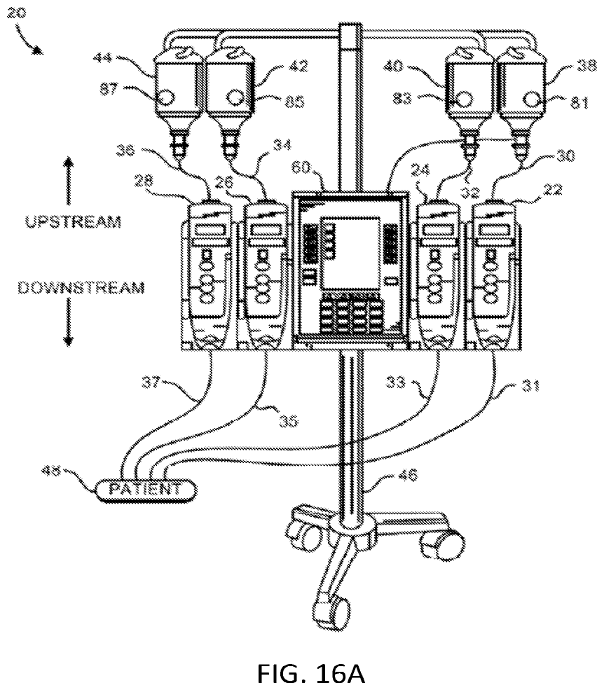

FIG. 14 depicts a flow diagram for detecting an occlusion using a sensor assembly, in accordance with some example implementations;

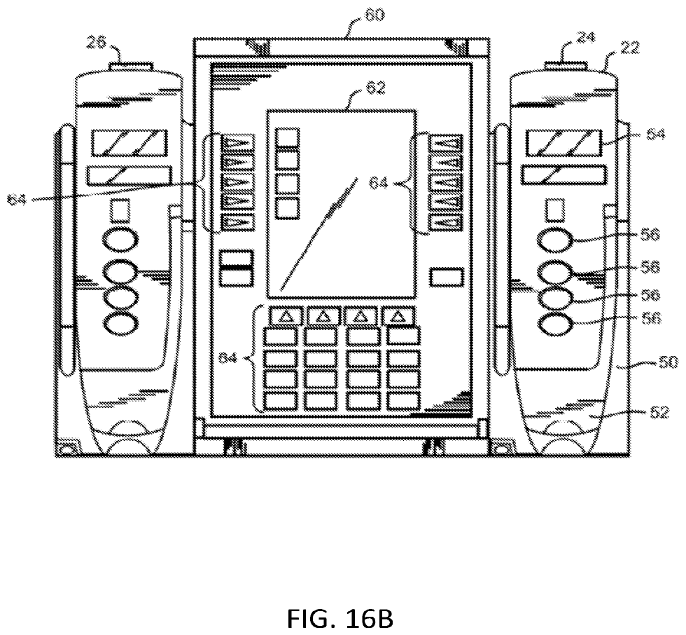

FIG. 15 depicts a block diagram illustrating a computing system, in accordance with some example implementations;

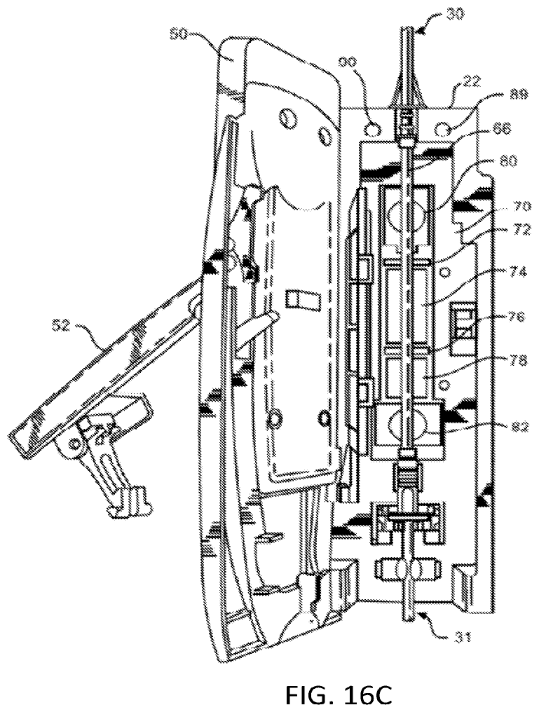

FIG. 16A depicts a front view of a patient care system, in accordance with some example implementations;

FIG. 16B depicts an enlarged view of a portion of a patient care system, in accordance with some example implementations; and

FIG. 16C depicts a perspective view of a pump, in accordance with some example implementations.

When practical, similar reference numbers denote similar structures, features, or elements.

### DETAILED DESCRIPTION

Fluid pumps, such as infusion pumps, administer therapy to patients by delivering a medication or other fluid to the patient. The fluid pumps may be connected with a fluid delivery tube, such as intravenous tubing or other administration sets. Various parameters of the fluid delivery by the fluid pumps may be adjusted based on the fluid pressure of the fluid passing through the fluid delivery tube to help ensure that a proper amount of fluid is delivered to the patient. During use of the fluid pumps, movement of the patient, or other sources may cause the fluid pressure within the fluid delivery tube to fluctuate, which if left unmonitored, may lead to unintended bolus, under-delivery, and/or cessation of the fluid delivered to the patient, thereby causing various medical complications. In some instances, at least a portion of the fluid passageway between the fluid storage and the patient may become occluded, which may impact pumping accuracy and may similarly lead to under-delivery and/or cessation of the fluid delivered to the patient, thereby causing various medical complications. Determining the changes in the fluid pressure, adjusting the pump to account for those changes, and detecting occlusions may be difficult. For example, sensors and detection methods for quickly measuring the fluid pressure, such as direct flow-rate measurements, may generally be unreliable, inaccurate, and require a significant amount of device resources (e.g., power, processing time, memory, network bandwidth, and the like), especially for low flow rates of the fluid delivered to the patient.

The infusion system described herein may employ an interlocked sensor assembly to improve the accuracy in detecting the fluid pressure of the fluid flowing along the fluid passageway and changes in the fluid pressure. Thus, the interlocked sensor assembly can be used to quickly and accurately detect undesirable changes in fluid pressure, occlusions along the fluid passageway, and/or the like. The interlocked sensor assembly may form a part of or be coupled to an infusion device to accurately detect a fluid pressure. For example, the interlocked sensor may include a body (which may form a part of or be coupled to an infusion device), a pressure diaphragm or flexible membrane, and a sensor mechanically interlocked with the pressure diaphragm. The flow of fluid through the fluid passageway causes movement of the pressure diaphragm. The movement of the pressure diaphragm in turn generates a load that is measured by the sensor. Since the load linearly corresponds to the fluid pressure of the fluid flowing along the fluid passageway, the measured load may be translated to accurately detect the fluid pressure of the fluid.

The interlocked connection between the sensor and the pressure diaphragm allows for a single sensor to be employed, allows for both negative and positive fluid pressure sensing, reduces stress on the components of the sensor assembly, may improve the usability of the sensor assembly, and may be reusable. As a result, the infusion system consistent with implementations of the current subject matter may provide an easy to use and improved system for fluid pressure detection, while reducing or eliminating unintended boluses, under-delivery, and/or cessation of fluid delivered to the patient.

In some instances, infusion devices, such as cassette-based infusion devices, can be very large and can require multiple sensors to detect the fluid pressure of the fluid flowing through the infusion device. In particular, cassette-based infusion devices generally include two cycles-a fill cycle, during which a fluid chamber is filled with a fluid, and a delivery cycle, during which the fluid is delivered to a patient-to deliver fluid to the patient. It may be desirable to increase the speed of the fill cycle to produce better flow continuity. However, increasing the speed of the fill cycle can create a large amount of pressure signal noise. Thus, measurements of the static line pressure may be inaccurate or may include a significant amount of noise. Additionally, sensors generally fail to accurately measure the fluid pressure within a desired response time. This can be problematic as the fill cycles can be very short. The interlocked sensor assembly consistent with implementations of the current subject matter may quickly and accurately detect the fluid pressure of the fluid within the fluid passageway.

To measure the fluid pressure within the fluid passageway, infusion devices may use two (or more) sensors-a first pressure sensor upstream of the infusion device to measure the upstream pressure, and a second pressure sensor downstream of the infusion device to measure the downstream pressure. However, multiple pressure sensors may lead to sensitive and inaccurate pressure measurements due at least in part to the increased noise and changes in fluid pressure. The sensor assembly consistent with implementations of the current subject matter allows for a single sensor to be used for measuring both upstream and downstream fluid pressures and for improving the accuracy of fluid pressure measurements.

In some infusion device configurations, an elastic member is used between a sensing element and the fluid. The elastic member allows the fluid pressure to produce a reaction force on the sensing element. In such configurations, negative fluid pressure pulls the elastic member away from the sensing element, reducing or eliminating contact between the elastic member and the sensing element. As a result, in such instances, the sensing element is unable to measure the negative fluid pressure, leading to an inaccurate representation of the fluid pressure.

When using such configurations to measure negative pressure, a preload force, higher than the reaction force the membrane produces when the negative pressure pulls the elastic member away from the sensing element, is needed. However, the preload force depends on a number of factors, such as the distance between the elastic member and the sensing element, the spring rate of the elastic member, the geometry of the elastic member, and the material properties of the elastic member. Thus it may be difficult to determine the appropriate preload force. Also, the preload force generally causes stress and strain in the material of the elastic member, leading to degradation of the material, extreme stress relaxation in the material over time, and inaccurate fluid pressure measurements. Consistent with implementations of the current subject matter, the interlocked sensor assembly eliminates the need for a preload force to measure negative pressures and can allow for the sensor to detect much lower negative pressures, providing accurate representations of the fluid pressure.

For proper filling and pumping of fluid in some infusion devices, a height differential is used between the fluid storage and the infusion device. In such configurations, fluid inflow into the infusion device may be reduced by inadequate fluid container head height or falling pressure levels within the fluid storage as the fluid storage is emptied. Additionally, restricted emptying of the fluid container by inadequate venting or an obstructed fluid path from an occlusion may also reduce flow. The interlocked sensor assembly consistent with implementations of the current subject matter may be insensitive to the positioning of the fluid storage relative to the infusion device and/or to the sensor. For example, the interlocked connection between the pressure diaphragm and the sensor provides a direct datum interface between the sensor and the pressure diaphragm, improving the accuracy of the fluid pressure measurements and reducing or eliminating the impact of the positioning of the fluid storage on the fluid pressure measurements.

Generally, in-line sensors or other sensors may be difficult to properly couple to the fluid passageway and may become dislodged. Misaligned sensors or improperly arranged sensors may lead to inaccurate fluid pressure measurements, which can be dangerous and harmful to the patient. The interlock connection between the pressure diaphragm and the sensor, consistent with implementations of the current subject matter, may provide an easy and effective mechanism for coupling the sensor to the pressure diaphragm of the sensor assembly. For example, the interlock connection allows for the sensor to be properly aligned and quickly coupled to the pressure diaphragm, thereby reducing the risk of improperly connected sensors and reducing or eliminating harm to the patient.

Moreover, in some infusion systems, such as infusion systems using multiple sensors or in-line sensors, such as when the sensors are in contact with the fluid flowing through the infusion system, the sensors may need to be disposed of, along with the infusion sets, after each use. Such systems may thus waste resources and result in significant expense. The interlocked sensor assembly, consistent with implementations of the current subject matter, may include a reusable sensor. For example, the sensor described herein may not contact the fluid to detect the fluid pressure. Instead, the sensor is coupled to the pressure diaphragm via an interlock connection such that the sensor does not contact the fluid and may be removed from the pressure diaphragm. Thus, even when the pressure diaphragm may be cleaned or disposed of, the sensor may be removed and reused by interlocking the sensor with another pressure diaphragm. Accordingly, the sensor assembly described herein may reduce waste.

FIG. 1 depicts a system diagram illustrating an infusion system 100, in accordance with some example implementations. Referring to FIG. 1, the infusion system 100 may include a fluid storage 120, a fluid infusion set 112 that includes an infusion device (also referred to herein as a “pump”) 122, a controller 108, and a sensor assembly 110, an upstream fluid delivery tube 106 connecting the fluid storage 120 and the fluid infusion set 112, a downstream fluid delivery tube 107 connecting the fluid infusion set 112 and a patient, a network 105, an accessory system 102, and a display 114. In some example implementations, the display 114, the controller 108, the sensor assembly 110, and/or the accessory system 102 may form a portion of the infusion device 122 and/or may be positioned within or on a housing of the infusion device 122. In some example implementations, the display 114, the controller 108, the sensor assembly 110, and/or the accessory system 102 may form a portion of the infusion device 122 and/or may be separately coupled to the infusion device 122.

For example, the display 114 may form a part of the infusion device 122 or may be separately coupled as part of a client device. The display 114 may also include a user interface. The user interface may form a part of a display screen of the display 114 that presents information to the user and/or the user interface may be separate from the display screen. For example, the user interface may be one or more buttons, or portions of the display screen that is configured to receive an entry from the user. The client device may be a mobile device such as, for example, a smartphone, a tablet computer, a wearable apparatus, and/or the like. However, it should be appreciated that the client device may be any processor-based device including, for example, a desktop computer, a laptop computer, a workstation, and/or the like. Via the display 114, the user may be able to configure certain parameters of the infusion device 122, a fluid pressure threshold, a desired flow rate or flow rate limit, an alarm limit, a start time of the infusion, an end time of the infusion, a length of time of a fill cycle of the infusion device, a length of time of a delivery cycle of the infusion device, and/or the like. Additionally, in some examples, via the display 114, the user may configure various fluid protocols with default settings and safety parameters (e.g., setting a limit to a dose of a fluid).

The accessory system 102 may include an alarm, light (e.g., an LED), a sound source, and/or other indicator. The indicator may indicate to the user of one or more measurements, thresholds, or other detected events relating to the infusion device 122. For example, the indicator may indicate to the user that a fluid pressure within a fluid passageway between the fluid storage and the patient is greater than, less than, or equal to a threshold, a rate of change of the fluid pressure is greater than, less than, or equal to a threshold, the fluid pressure does not match a desired fluid pressure, an occlusion, such as a blockage, a presence of air, and an obstruction, occurred along the fluid passageway, and the like. As noted above, the accessory system 102 may form a part of the infusion device 122 and/or the display 114, or may be separately coupled to the infusion device 122 and/or the display 114, such as via the network 105.

As FIG. 1 shows, the infusion set 112 (e.g., the infusion device 122, controller 108, and/or the sensor assembly 110), the display 114, and/or the accessory system 102 may be communicatively coupled via a network 105. The network 105 may be any wired and/or wireless network including, for example, a public land mobile network (PLMN), a local area network (LAN), a virtual local area network (VLAN), a wide area network (WAN), the Internet, and/or the like.

The infusion device 122 may be any type of pump configured to move a fluid from a fluid storage 120, such as a reservoir, drip chamber, syringe, and/or the like, through a conduit or other tube, such as the upstream fluid delivery tube 106 and the downstream fluid delivery tube 107, to a destination, such as, for example, a patient. The infusion device 122 may be a Large Volume Infusion Pump (LVP), a syringe pump (SP), an anesthesia delivery pump, infusion pump, a patient-controlled analgesic (PCA) pump, a cassette-based pump, an ambulatory pump, a delivery system using a flow stop to control the flow of fluid, and/or the like configured to deliver a medication to a patient. However, it should be appreciated that the infusion device 122 may be any infusion device configured to deliver a substance (e.g., fluid, nutrients, medication, and/or the like) to a patient's circulatory system or epidural space via, for example, intravenous infusion, subcutaneous infusion, arterial infusion, epidural infusion, and/or the like. Additionally and/or alternatively, the infusion device 122 may be an infusion device configured to deliver a substance (e.g., fluid, nutrients, medication, and/or the like) to a patient's digestive system via a nasogastric tube (NG), a percutaneous endoscopic gastrostomy tube (PEG), nasojejunal tube (NJ), and/or the like. Moreover, the infusion device 122 may be part of a patient care system (e.g., the patient care system 20) that includes one or more additional pumps.

The infusion device 122 may include or may be coupled to a controller 108. The controller 108 may determine and/or control a fluid flow rate, such as a level of the fluid flow rate, changes in the fluid flow rate, and/or the like. For example, in some implementations, the controller 108 receives a desired value and/or rate of change of the fluid flow rate selected via the display 114. In some implementations, the controller 108 may communicate with one or more other systems, such as the accessory system 102, the display 114, and/or the sensor assembly 110.

FIG. 2 schematically depicts a top cross-sectional view of an example sensor assembly 110, consistent with implementations of the current subject matter. The sensor assembly 110 includes a body 152, a pressure diaphragm 154, and a sensor 150. The sensor assembly 110 may also include a fluid passageway 140 formed at least in part by the body 152 and the pressure diaphragm 154. The fluid passageway 140 may also include the fluid delivery tubes 106, 107, and/or may extend through other portions of the body 152.

The sensor 150 may include a force sensor, a pressure sensor, or the like. For example, the sensor 150 may measure a force or a load generated by movement of the pressure diaphragm 154 as a result of fluid pressure on the pressure diaphragm 154 (as described in more detail below). The sensor 150 may make fluid pressure readings continuously and/or at various time intervals (e.g., every 10 seconds, 30 seconds, 1 minute, 30 minutes, 1 hour, 12hours, 24 hours, and the like). In some implementations, the controller 108 controls the time at which the sensor 150 measures the fluid pressure. The sensor 150 may transmit the load readings to the controller 108 and/or to the infusion device 122 (e.g., the controller 108).

The controller 108 may translate or convert the load measurements to determine the fluid pressure of the fluid flowing through the fluid passageway. For example, as shown in the graph 600 in FIG. 6, the measured load has a linear relationship with the fluid pressure of the fluid flowing through the fluid passageway. Accordingly, the controller 108 may receive the load readings from the sensor 150 and convert the load readings to fluid pressure measurements. In some implementations, the controller 108 detects an occlusion along the fluid passageway 140 based at least in part on one or more sensor readings from the sensor 150 of the sensor assembly 110. For example, the controller 108 may compare a detected fluid pressure to a threshold pressure (e.g., 1 psi, 2 psi, 1 to 2 psi, 2 to 3 psi, 3 to 4 psi, and/or the like). The controller 108 may detect the occlusion when the detected fluid pressure is less than or equal to the threshold pressure.

In some example implementations, the controller 108 may respond to the detection of the occlusion by generating a log whose content may include, for example, a time, a date, and/or other information to facilitate review of the infusion set 112. In some implementations, the controller 108 may respond to the detection by triggering an indicator, such as a visual indicator on a graphical user interface (e.g., the display 114), to indicate that an occlusion was detected. In some implementations, the controller 108 may adjust a delivery workflow of the infusion device 122 after detecting the occlusion. For example, the controller 108 may prohibit additional fluid from being delivered to the patient until a reset code or authorization for delivering the fluid is received. In some implementations, the controller 108 may adjust one or more settings of the infusion device 122, based at least in part on the determined fluid pressure and/or detection of the occlusion. For example, the controller 108 may adjust one or more settings of the infusion device 122, such as a speed of the fluid flow or delivery rate, an amount of fluid delivered to the patient, a type of fluid delivered to the patient and/or the like.

Referring back to FIG. 2, the sensor 150 may be interlocked with or otherwise have an interlocked connection with the pressure diaphragm 154, which is in turn coupled to the body 152. For example, the sensor 150 may include a sensor interface 160 (described in more detail below) that is interlocked with or has an interlocked connection with a corresponding diaphragm interface 170 of the pressure diaphragm 154.

The body 152 may be coupled to (e.g., directly coupled to, fluidly coupled to, etc.) the infusion device 122 and/or may form a portion of the infusion device 122. For example, the body 152 may include or form a part of a cassette for the infusion device 122. Additionally or alternatively, the body 152 is a passive element without electronics or internal electronic circuitry. In other words, the body 152 may not actively monitor and/or control one or more aspects of delivering fluid to the patient.

The body 152 may include a body stop 156. The body stop 156 may extend from a base portion 158 of the body 152 into an interior volume of the pressure diaphragm 154. The body stop 156 may prevent the pressure diaphragm 154 from collapsing into the base portion 158 of the body 152, such as during loading.

The pressure diaphragm 154 may be coupled to the body 152. The pressure diaphragm 154 may define a flexible membrane. For example, the pressure diaphragm 154 may include an elastomeric material, such as thermoplastic elastomer, silicone, polyurethane, and/or the like. The pressure diaphragm 154 may move as a function of fluid pressure on the inner side of the pressure diaphragm 154. The movement of the pressure diaphragm 154 generates a load on the sensor 150, which as noted above, can be used to determine the fluid pressure of the fluid flowing through the fluid passageway.

For example, the interlocked connection between the pressure diaphragm 154 and the sensor 150 is configured to transfer a load from the pressure diaphragm 154 to the sensor 150 where the load indicates the fluid pressure of the fluid passing through the fluid passageway 140 between the pressure diaphragm 154 and the body 152. The fluid passing through the fluid passageway 140 causes the pressure diaphragm 154 to move from a first position to a second position. The movement of the pressure diaphragm 154 from the first position to the second position is configured to generate the load measured by the sensor 150. In some implementations, the first position is an expanded position and the second position is a compressed position (or vice versa). The fluid pressure may be a negative pressure when the pressure diaphragm 154 is in the compressed position and the fluid pressure may be a positive pressure when the pressure diaphragm 154 is in the expanded position. As described herein, the sensor 150 may measure the negative pressure without the use of a preload force, reducing stress and strain on the pressure diaphragm 154.

Referring to FIG. 2, the pressure diaphragm 154 includes an inner side 172 and an outer side 174. The inner side 172 is configured to face towards the body 152. The inner side 172 may surround an interior volume 176 of the pressure diaphragm 154. The interior volume 176 is configured to receive at least a portion of the fluid flowing through the fluid passageway. In other words, at least a portion of the fluid passageway 140 is formed between the inner side 172 and the body 152. The outer side 174 is opposite the inner side 172. The outer side 174 includes the diaphragm interface 170.

Referring again to FIG. 2, the pressure diaphragm 154 includes a base portion 180, a central portion 182, and an interface portion 184. The base portion 180 is configured to be coupled to the body 152 of the sensor assembly 110. The base portion 180 defines a flange. The flange may extend radially outwardly from the central portion 182 to provide a surface suitable for coupling to the body 152. The base portion 180 may seal the interior volume 176 to prevent leakage of the fluid from the fluid passageway.

The central portion 182 extends radially outwardly from the base portion 180. The central portion 182 may include a flexible feature, such as a fold (e.g., an accordion-type fold), a curve, and/or the like. The central portion 182 may be flexible to permit movement of the pressure diaphragm 154 in a radially outwardly and/or lateral direction. For example, the central portion 182 may allow for movement (e.g., expansion, compression, and the like) of the pressure diaphragm 154 away from and/or towards the body 152. The central portion 182 may additionally or alternatively allow for movement of at least the interface portion 184 in a lateral direction, such as a direction along a central axis that is approximately perpendicular to a direction of flow of the fluid through the fluid passageway. Accordingly, via the central portion 182, the movement of the pressure diaphragm 154 may generate a load that is transferred to the sensor 150 interlocked with the pressure diaphragm 154.

The interface portion 184 includes the diaphragm interface 170 that is the interlocked connection with the sensor interface 160 of the sensor 150. The diaphragm interface 170 may generally have a crown shape that matches and engages with the corresponding sensor interface 160 of the sensor 150. For example, the diaphragm interface 170 includes a diaphragm mating feature 186 and the sensor interface 160 includes a sensor mating feature 188 that corresponds to the diaphragm mating feature 186. The diaphragm mating feature 186 may include one or more of a first shape, a first angle, a first edge, and/or the like and the sensor mating feature 188 may include one more of a second shape, a second angle, a second edge, and/or the like that corresponds to a respective one of the first shape, the first angle, the first edge, and/or the like.

In some implementations, the diaphragm interface 170 defines a receptacle 191 that receives and mates with the sensor interface 160. The receptacle 191 is surrounded by a flat mating surface 193 that is configured to mate with a corresponding surface of the sensor 150. Referring to FIG. 2, within the receptacle, the diaphragm mating feature 186 includes an outer surface 190, a central surface 192, and/or an inner surface 194, though other mating configurations are contemplated. The sensor mating feature 188 includes an inner surface 190A, a central surface 192A, and/or an outer surface 194A that has a corresponding shape, size, and/or angle to the respective outer surface 190, central surface 192 and/or inner surface 194.

The outer surface 190, the central surface 192, and the inner surface 194 may together define an interior surface of the receptacle 191. In some implementations, each of the outer surface 190, the central surface 192, and the inner surface 194 are configured to contact and/or interlock with a corresponding surface of the sensor 150.

The outer surface 190 extends from the flat mating surface 193. The outer surface 190 extends at an angle from the flat mating surface 193 in a radially outwardly direction, towards the base portion 180 and/or the central portion 182, to the central surface 192. The central surface 192 extends at an angle from the outer surface 190 in a radially inwardly direction, towards the base portion 180 and/or the central portion 182, to the inner surface 194. The inner surface 194 may be flat. The inner surface 194 may form at least a portion of a separator 178 that separates the diaphragm interface 170 from the interior volume 176 of the pressure diaphragm 154. Such configurations allow for the sensor 150 to be interlocked with the pressure diaphragm 154 and to maintain contact with the pressure diaphragm 154. In other words, such configurations may allow for the sensor 150 to be snapped into the receptacle 191 and to be retained by the diaphragm mating feature 186. In some implementations, when the sensor assembly 110 is coupled to the infusion device 122, the diaphragm mating feature 186 (e.g., the diaphragm interface 170) expands radially to allow the sensor interface 160 to engage the diaphragm mating feature 186. Thus, the interlocked connection between the pressure diaphragm 154 and the sensor 150 improves the accuracy of the detection of the fluid pressure.

FIGS. 3-5 illustrate an example of the sensor assembly 110 consistent with implementations of the current subject matter. The example sensor assembly 110 shown in FIGS. 3-5 may be deployed in an infusion device 122, such as a piston-type or cassette-based infusion device 122. In such configurations, the body 152 may form a part of a cassette of the infusion device 122. The cassette may include a fluid chamber and a pumping mechanism. The fluid chamber may be defined at least in part by the interior volume 176 of the pressure diaphragm 154. The fluid chamber may store an amount of the fluid to be delivered to the patient and may form at least a part of the fluid passageway. The pumping mechanism may cause the fluid from within the fluid chamber to flow through the fluid passageway 140 to be delivered to the patient.

The pumping mechanism of the cassette may include a piston. The piston is configured to allow fluid to enter the fluid chamber (e.g., from the upstream fluid delivery tube 106) and/or to cause the fluid from the fluid chamber to be delivered to the patient (e.g., through the downstream fluid delivery tube 107). The piston may slide within a piston channel 199 of the body 152 of the sensor assembly 110. In some implementations, the piston opens and/or closes one or more valves coupled to the infusion device 122 to allow fluid to fill the fluid chamber and to cause the fluid from the fluid chamber to be delivered to the patient.

For example, the pumping mechanism may operate in a first state, such as a fill cycle, in which the pumping mechanism allows fluid to fill the fluid chamber. The pumping mechanism may additionally or alternatively operate in a second state, such as a delivery cycle, in which the pumping mechanism causes the fluid to flow from the fluid chamber to the patient. The pumping mechanism is configured to alternate between operation in the first state and operation in the second state to fill the fluid chamber and to deliver the fluid to the patient. Movement of the fluid (e.g., fluid pressure of the fluid) within and/or through the fluid chamber causes at least a portion of the pressure diaphragm 154 to move. Movement of the pressure diaphragm 154 generates a load or force that is measured by the sensor 150. As described herein, the controller 108 and/or the sensor 150 may translate the measured load into a fluid pressure of the fluid.

FIGS. 7-12 illustrate an example of the sensor assembly 110 consistent with implementations of the current subject matter. The example sensor assembly 110 shown in FIGS. 7-12 may be coupled (e.g., directly, fluidly, and the like) to the infusion device 122, such as a flow stop 122A. The flow stop 122A may allow the fluid to flow through the fluid passageway, from the upstream fluid delivery tube 106, to be delivered to the patient, through the downstream fluid delivery tube 107. For example, the flow stop 122A may be opened and closed to permit and/or prevent the flow of fluid to the patient. In some implementations, the sensor assembly 110 is used with other infusion devices, such as a peristaltic mechanism that allows the fluid to flow through the fluid passageway, from the upstream fluid delivery tube 106, to be delivered to the patient, through the downstream fluid delivery tube 107.

For example, the flow stop 122A may be manually or automatically (e.g., by the controller 108) manipulated to allow fluid to fill a pumping segment 106A of the upstream fluid delivery tube 106. For example, the flow stop 122A may be closed to allow fluid to fill the pumping segment 106A and to prevent the delivery of the fluid to the patient. The flow stop 122A may also be manually or automatically (e.g., by the controller 108) manipulated to allow fluid to be delivered to the patient. For example, the flow stop 122A may be opened to allow fluid to pass from the pumping segment 106A, through the fluid passageway 140 within the body 152, and through the downstream fluid delivery tube 107 to the patient. Movement of the fluid (e.g., fluid pressure of the fluid) within and/or through the fluid passageway 140 within the body 152 and across the fluid chamber causes at least a portion of the pressure diaphragm 154 to move. Movement of the pressure diaphragm 154 generates a load or force that is measured by the sensor 150. As described herein, the controller 108 and/or the sensor 150 may translate the measured load into a fluid pressure of the fluid.

Referring to FIGS. 9 and 10, the sensor assembly 110 may additionally and/or alternatively include a pressure diaphragm retainer 155. The pressure diaphragm retainer 155 may include a central opening 157 that allows at least the diaphragm interface 170 to pass therethrough. The pressure diaphragm retainer 155 may be coupled to the body 152, such as via a mechanical coupling, a snap-fit configuration, an adhesive, or the like, to secure the pressure diaphragm 154 to the body 152. For example, the base portion of the pressure diaphragm 154 may be positioned between the pressure diaphragm retainer 155 and the body 152 when the pressure diaphragm retainer 155 is coupled to the body 152.

FIGS. 13A and 13B show an example of the infusion set 112 shown in FIGS. 7-12 loaded onto an infusion device 122. In this configuration, the infusion device 122 may be configured to open and close the flow stop 122A to allow or prevent fluid from passing through the fluid passageway.

A method of manufacturing the infusion set 112 is also contemplated. The method includes coupling a pressure diaphragm to a body. The pressure diaphragm may include a diaphragm interface configured to interface with a sensor interface of a sensor in an interlocked connection. The interlocked connection is configured to transfer a load from the pressure diaphragm to the sensor. The coupling forms a fluid passageway between the pressure diaphragm and the body. The load indicates a fluid pressure of fluid passing through the fluid passageway. The method may also include coupling a fluid pumping segment to a first portion of the body. The method may further include coupling a fluid delivery tube to a second portion of the body. The fluid passageway is configured allow the fluid to pass from the fluid pumping segment through the fluid passageway to the fluid delivery tube. The coupling may include bonding elements using an adhesive or integrally molding items to form a unified element. In some implementations, the coupling may include a coupling device such as a valve or a connector.

FIG. 14 depicts a flowchart illustrating a process 1400 for detecting an occlusion in a fluid passageway, consistent with implementations of the current subject matter.

At 1402, a controller, such as the controller 108, may cause fluid to flow through a fluid passageway, such as the fluid passageway 140, of a sensor assembly, such as the sensor assembly 110 for an infusion device, such as the infusion device 122. The sensor assembly may include a body, such as the body 152, a pressure diaphragm, such as the pressure diaphragm 154, and a sensor, such as the sensor 150. The pressure diaphragm may be coupled to the body and include a diaphragm interface, such as the diaphragm interface 170. The sensor may include a sensor interface, such as the sensor interface 160. The sensor interface may be coupled to the diaphragm interface in an interlocked connection. The interlocked connection may transfer a load from the pressure diaphragm to the sensor. Consistent with implementations of the current subject matter, movement of the pressure diaphragm, caused by the flow of fluid through the fluid passageway, may generate the load. The sensor may measure the load transferred to the sensor by the pressure diaphragm. The sensor may then transmit the measured load to the controller. In other words, the controller may receive the measured load from the sensor. In other implementations, the sensor converts the load to a fluid pressure and transmits the fluid pressure to the controller.

At 1404, the controller determines, based on the measured load, a fluid pressure of the fluid. For example, the measured load has a linear relationship with the fluid pressure of the fluid flowing through the fluid passageway. Accordingly, the controller may receive the load readings from the sensor and convert the load readings to fluid pressure measurements.

At 1406, the controller may detect, based on a comparison of the fluid pressure to a threshold pressure, an occlusion in the fluid passageway. For example, the controller may determine that the fluid pressure is less than or equal to the threshold pressure. The threshold pressure may be 1 psi, 2 psi, 1 to 2 psi, 2 to 3 psi, 3 to 4 psi, and/or the like. The determination that the fluid pressure is less than or equal to the threshold pressure indicates that a blockage, a presence of air, and/or another obstruction exists along the fluid passageway, which can be harmful to a patient and lead to unintended bolus, under-delivery, and/or cessation of the fluid delivered to the patient, causing various medical complications.

At 1408, the controller may prevent, based on the detection of the occlusion, delivery of the fluid to a patient. For example, the controller may send a signal to the infusion device to cause the infusion device to stop delivery of the fluid to the patient. The controller may additionally or alternatively change one or more parameters of the infusion device upon detection of the occlusion. For example, the controller may change a flow rate of the fluid through the fluid passageway, start or stop a fill cycle, and/or start or stop a delivery cycle.

In some implementations, the controller transmits an alert, such as to a display (e.g., the display 114). The alert may include a visual, audio, audiovisual, tactile, and/or the like, indicator that indicates the presence of an occlusion. The alert may signal to a physician that delivery of the fluid to the patient should be stopped, there is an issue along the fluid passageway, and/or the like.

In some implementations, the controller may adjust one or more settings of the infusion device, based at least in part on the determined fluid pressure. For example, the controller may adjust one or more settings of the infusion device, such as a speed of the fluid flow or delivery rate, an amount of fluid delivered to the patient, a type of fluid delivered to the patient and/or the like. The infusion system including the sensor assembly described herein may desirably accurately adjust the one or more settings of the infusion device based on more accurate fluid pressure determinations, which helps to improve control of the infusion device and reduce or eliminate unintended boluses, under-delivery, and/or cessation of fluid flow within the fluid passageway due to an occlusion or other changes in the fluid pressure.

FIG. 15 depicts a block diagram illustrating a computing system 500 consistent with implementations of the current subject matter. Referring to FIGS. 1 and 15, the computing system 500 can be used to implement the infusion set 112, the accessory system 102, the display 114, and/or any components therein.

As shown in FIG. 15, the computing system 500 can include a processor 510, a memory 520, a storage device 530, and input/output devices 540. The processor 510, the memory 520, the storage device 530, and the input/output devices 540 can be interconnected via a system bus 550. The processor 510 is capable of processing instructions for execution within the computing system 500. Such executed instructions can implement one or more components of, for example, the infusion device 122. In some example implementations, the processor 510 can be a single-threaded processor. Alternatively, the processor 510 can be a multi-threaded processor. The processor 510 is capable of processing instructions stored in the memory 520 and/or on the storage device 530 to present graphical information for a user interface provided via the input/output device 540.

The memory 520 is a computer readable medium such as volatile or non-volatile that stores information within the computing system 500. The memory 520 can store data structures representing configuration object databases, for example. The storage device 530 is capable of providing persistent storage for the computing system 500. The storage device 530 can be a floppy disk device, a hard disk device, an optical disk device, or a tape device, or other suitable persistent storage means. The input/output device 540 provides input/output operations for the computing system 500. In some example implementations, the input/output device 540 includes a keyboard and/or pointing device. In various implementations, the input/output device 540 includes a display unit for displaying graphical user interfaces.

According to some example implementations, the input/output device 540 can provide input/output operations for a network device. For example, the input/output device 540 can include Ethernet ports or other networking ports to communicate with one or more wired and/or wireless networks (e.g., a local area network (LAN), a wide area network (WAN), the Internet).

In some example implementations, the computing system 500 can be used to execute various interactive computer software applications that can be used for organization, analysis and/or storage of data in various formats. Alternatively, the computing system 500 can be used to execute software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 540. The user interface can be generated and presented to a user by the computing system 500 (e.g., on a computer screen monitor, etc.).

In some example implementations, the infusion device 122 (e.g., pump 22 as shown in FIGS. 16A-16C) may be part of a patient care system 20. FIGS. 16A-16C illustrate example implementations of the patient care system 20, though other types of patient care systems may be implemented. Referring to FIG. 16A, the patient care system 20 may include the pump 22 as well as additional pumps 24, 26, and 28. Although a large volume pump (LVP) is illustrated, other types of pumps may be implemented, such as a small volume pump (SVP), a syringe pump, an anesthesia delivery pump, and/or a patient-controlled analgesic (PCA) pump configured to deliver a medication to a patient. The pump 22 may be any infusion device configured to deliver a substance (e.g., fluid, nutrients, medication, and/or the like) to a patient's circulatory system or epidural space via, for example, intravenous infusion, subcutaneous infusion, arterial infusion, epidural infusion, and/or the like, or the pump 22 may be an infusion device configured to deliver a substance (e.g., fluid, nutrients, medication, and/or the like) to a patient's digestive system via a nasogastric tube (NG), a percutaneous endoscopic gastrostomy tube (PEG), nasojejunal tube (NJ), and/or the like.

As shown in FIG. 16A, each of the pump 22, 24, 26, and 28 may be fluidly connected with an upstream fluid line 30, 32, 34, and 36, and/or the upstream fluid delivery tube 106, respectively. Moreover, each of the four pumps 22, 24, 26, and 28 may also fluidly connected with a downstream fluid line 31, 33, 35, and 37, and/or the downstream fluid delivery tube 107, respectively. The fluid lines can be any type of fluid conduit, such as fluid delivery tube (e.g., the fluid delivery tube 106, the fluid delivery tube 107, etc.), through which fluid can flow. At least a portion of one or more of the fluid lines may be constructed with a multi-layered configuration as described herein. In some implementations, each of the pumps 22, 24, 26, and 28 may use the same fluid line. In such implementations, as described above, the infusion system may detect when various types of fluid flow through the fluid line. In some implementations, the pump 22 may be coupled to a sensor assembly, such as the sensor assembly 110, which may help to detect a fluid pressure of the fluid flowing through the fluid lines. This helps to improve reduce unintended boluses, under-delivery, and/or cessation of fluid delivered to the patient.

Fluid supplies 38, 40, 42, and 44 (such as the fluid storage 120), which may take various forms but in this case are shown as bottles, are inverted and suspended above the pumps. Fluid supplies may also take the form of bags, syringes, or other types of containers. Both the patient care system 20 and the fluid supplies 38, 40, 42, and 44 may be mounted to a roller stand or intravenous (IV) pole 46.

A separate pump 22, 24, 26, and 28 may be used to infuse each of the fluids of the fluid supplies into the patient. The pumps 22, 24, 26, and 28 may be flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may comprise drugs or nutrients or other fluids.

Typically, medical fluid administration sets have more parts than are shown in FIG. 16A. Many have check valves, drip chambers, valved ports, connectors, and other devices well known to those skilled in the art. These other devices have not been included in the drawings so as to preserve clarity of illustration. In addition, it should be noted that the drawing of FIG. 16A is not to scale and that distances have been compressed for the purpose of clarity. In an actual setting, the distance between the fluid supplies 38, 40, 42, and 44 and the pump modules 22, 24, 26, and 28 could be much greater.

Referring now to FIG. 16B, an enlarged view of the front of the patient care system 20 is shown. The pump 22 may include a front door 50 and a handle 52 that operates to lock the door in a closed position for operation and to unlock and open the door for access to the internal pumping and sensing mechanisms and to load administration sets for the pump. When the door is open, the tube (e.g., the fluid delivery tube 106) can be connected with the pump, as will be shown in FIG. 16C. When the door is closed, the tube is brought into operating engagement with the pumping mechanism, the upstream and downstream pressure sensors, and the other equipment of the pump. A display 54 (e.g., the display 114), such as an LED display, is located in plain view on the door in this embodiment and may be used to visually communicate various information relevant to the pump, such as alert indications (e.g., alarm messages). The display 54 may otherwise be a part of or be coupled to the pump 22. Control keys 56 exist for programming and controlling operations of the pump as desired. The pump 22 also includes audio alarm equipment in the form of a speaker (not shown).

In the embodiment shown, a programming module 60 is attached to the left side of the pump 22. In some implementations, the programming module 60 forms a part of the pump 22. Other devices or modules, including another pump, may be attached to the right side of the pump 22, as shown in FIG. 16A. In such a system, each attached pump represents a pump channel of the overall patient care system 20. In one embodiment, the programming module is used to provide an interface between the pump 22 and external devices as well as to provide most of the operator interface for the pump 22.

The programming module 60 includes a display 62 for visually communicating various information, such as the operating parameters of the pump 22 and alert indications and alarm messages. The programming module 60 may additionally and/or alternatively communicate with the accessory system 102 to for, example, indicate that the predicted motor pressure and the measured fluid pressure have not converged (e.g., the error value is not less than the error threshold after a threshold number of cycles). The programming module 60 may additionally and/or alternatively display on the display 54, one or more parameters of the fluid, such as the fluid pressure, the patient pressure, and/or the motor pressure of the fluid in the fluid delivery tube (e.g. the fluid delivery tube 106). The programming module 60 may also include a speaker to provide audible alarms. The programming module or any other module also has various input devices in this embodiment, including control keys 64 and a bar code or other scanner or reader for scanning information from an electronic data tag relating to the infusion, the patient, the care giver, or other. The programming module also has a communications system (not shown) with which it may communicate with external equipment such as a medical facility server or other computer and with a portable processor, such as a handheld portable digital assistant (“PDA), or a laptop-type of computer, or other information device that a care giver may have to transfer information as well as to download drug libraries to a programming module or pump.

The communications system may take the form of a radio frequency (“RF”) (radio frequency) system, an optical system such as infrared, a Bluetooth system, or other wired or wireless system. The bar code scanner and communications system may alternatively be included integrally with the pump 22, such as in cases where a programming module is not used, or in addition to one with the programming module. Further, information input devices need not be hard-wired to medical instruments, information may be transferred through a wireless connection as well.

FIG. 16B includes a second pump 26 connected to the programming module 60. As shown in FIG. 16A, more pump modules may be connected. Additionally, other types of modules may be connected to the pump modules or to the programming module.

Turning now to FIG. 16C, the pump 22 is shown in perspective view with the front door 50 open, showing the upstream fluid line 30 and downstream fluid line 31 in operative engagement with the pump 22. The pump 22 directly acts on a tube 66 (also referred to as a pump segment) that connects the upstream fluid line 30 to the downstream fluid line 31 to form a continuous fluid conduit, extending from the respective fluid supply 38 (FIG. 16A) to the patient 48, through which fluid is acted upon by the pump to move fluid downstream to the patient. Specifically, a pumping mechanism or flow control device 70 acts as the flow control device of the pump to move fluid though the conduit. The upstream and downstream fluid lines and/or tube 66 may be coupled to a pump cassette or cartridge that is configured to be coupled to the pump 22, such as the type described in co-pending U.S. patent application Ser. No. 13/827,775, which is incorporated by reference herein. Similarly, the sensor assembly described herein may be coupled to or form a part of the pump cassette or cartridge.

The type of pumping mechanism may vary and may be for example, a multiple finger pumping mechanism. For example, the pumping mechanism may be of the “four finger” type and includes an upstream occluding finger 72, a primary pumping finger 74, a downstream occluding finger 76, and a secondary pumping finger 78. The “four finger” pumping mechanism and mechanisms used in other linear peristaltic pumps operate by sequentially pressing on a segment of the fluid conduit by means of the cam-following pumping fingers and valve fingers 72, 74, 76, and 78. The pressure is applied in sequential locations of the conduit, beginning at the upstream end of the pumping mechanism and working toward the downstream end. At least one finger is always pressing hard enough to occlude the conduit. As a practical matter, one finger does not retract from occluding the fluid delivery tube until the next one in sequence has already occluded the fluid delivery tube; thus at no time is there a direct fluid path from the fluid supply to the patient. The operation of peristaltic pumps including four finger pumps is well known to those skilled in the art and no further operational details are provided here.

In this particular embodiment, FIG. 16C further shows a downstream pressure sensor 82 included in the pump 22 at a downstream location with respect to the pumping mechanism. The downstream pressure sensor 82 is mounted to the flow control device 70 and is located adjacent and downstream in relation to the flow control device. The downstream pressure sensor is located downstream from the flow control device, that is, at a location between the patient 48 (FIG. 16A) and the flow control device, so that the connection of the correct fluid supply with the correct pump may be verified before any fluid is pumped to the patient.

With reference still to FIG. 16C, an upstream pressure sensor 80 may also be included in the pump 22. The upstream pressure sensor is assigned to the flow control device or pumping mechanism 70 and, in this embodiment, is further provided as an integral part of the pump 22. It is mounted to the flow control device 70 and is located adjacent and upstream in relation to the flow control device. The upstream pressure sensor is located upstream from the flow control device, that is, at a location between the fluid supply 38 (FIG. 16A) and the flow control device, so that the connection of the correct fluid supply with the correct pump may be verified before any fluid is pumped to the patient. In an implementation where the source is a syringe, the flow control device 70 may be configured to press a plunger of the syringe to provide the infusion according to the programmed parameters. In some implementations, rather than a separate downstream pressure sensor and downstream pressure sensor, the pump 22 may include or may be coupled to the sensor assembly 110 consistent with implementations of the current subject matter, to accurately and quickly determine the fluid pressure of the fluid, using a single sensor.

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, NET™, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, or the like. A message may, in some implementations, include a signal utilized to transmit one or more representations of the information. While recited in the singular, it will be understood that a message may be composed, transmitted, stored, received, etc. in multiple parts.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1-63. (canceled)

64. A sensor assembly for an infusion system, the sensor assembly comprising:
a body;
a pressure diaphragm coupled to the body, the pressure diaphragm comprising a diaphragm interface;
a fluid passageway formed between the body and the pressure diaphragm, the fluid passageway configured to allow a fluid to pass therethrough to be delivered to a patient; and
a sensor comprising a sensor interface coupled to the diaphragm interface in an interlocked connection, the interlocked connection configured to transfer a load from the pressure diaphragm to the sensor, the load indicating a fluid pressure of the fluid passing through the fluid passageway.

65. The sensor assembly of claim 64, wherein the fluid passing through the fluid passageway is configured to cause the pressure diaphragm to move from a first position to a second position, the movement of the pressure diaphragm from the first position to the second position configured to generate the load.

66. The sensor assembly of claim 65, wherein the first position is an expanded position; and wherein the second position is a compressed position.

67. The sensor assembly of claim 65, wherein the first position is a compressed position; and wherein the second position is an expanded position.

68. The sensor assembly of claim 66, wherein the fluid pressure is a negative pressure when the pressure diaphragm is in the compressed position; and wherein the fluid pressure is a positive pressure when the pressure diaphragm is in the expanded position.

69. The sensor assembly of claim 64, wherein the pressure diaphragm further comprises:
an inner side; and
an outer side opposite the inner side, the outer side comprising the diaphragm interface;
wherein the fluid passageway is formed between the inner side and the body.

70. The sensor assembly of claim 64, wherein the pressure diaphragm defines a flexible membrane.

71. The sensor assembly of claim 69, wherein the inner side of the pressure diaphragm surrounds an interior volume of the pressure diaphragm.

72. The sensor assembly of claim 64, wherein the pressure diaphragm comprises:
a base portion coupled to the body;
a central portion; and
an interface portion comprising the diaphragm interface.

73. The sensor assembly of claim 72, wherein the central portion is flexible to permit movement of the pressure diaphragm in a radially outward direction.

74. The sensor assembly of claim 73, further comprising a body stop extending into an interior volume of the pressure diaphragm, the body stop configured to prevent the pressure diaphragm from collapsing when the sensor assembly is coupled to a fluid administration set.

75. The sensor assembly of claim 64, wherein the diaphragm interface comprises a diaphragm mating feature; and wherein the sensor interface comprises a sensor mating feature that corresponds to the diaphragm mating feature, and wherein the diaphragm mating feature comprises one or more of a first shape, a first angle, and a first edge; and wherein the sensor mating feature comprises one or more of a second shape corresponding to the first shape, a second angle corresponding to the first angle, and a second edge corresponding to the first edge.

76. The sensor assembly of claim 64, wherein the sensor interface defines a protrusion; and wherein the pressure diaphragm interface defines a receptacle configured to receive the protrusion.

77. The sensor assembly of claim 64, wherein the body forms a part of a cassette for the infusion system, the cassette comprising:
a fluid chamber defined at least in part by the pressure diaphragm, the fluid chamber configured to store an amount of the fluid; and
a pumping mechanism configured to cause the fluid from within the fluid chamber to flow through the fluid passageway to be delivered to the patient.

78. The sensor assembly of claim 77, wherein the pumping mechanism comprises a piston.

79. The sensor assembly of claim 77, wherein the pumping mechanism is configured to operate in a first state; wherein the pumping mechanism is configured to allow fluid to fill the fluid chamber in the first state.

80. The sensor assembly of claim 77, wherein the pumping mechanism is configured to operate in a second state; wherein the pumping mechanism is configured to cause the fluid to flow from the fluid chamber to the patient in the second state.

81. The sensor assembly of claim 64, wherein the body is coupled to a flow stop;
wherein operation of the flow stop is configured to one or more of: allow the fluid to flow through the fluid passageway for delivery to a patient and prevent the fluid from flowing through the fluid passageway for delivery to the patient.

82. The sensor assembly of claim 64, wherein the body is coupled to a peristaltic mechanism; wherein operation of the peristaltic mechanism is configured to one or more of: allow the fluid to flow through the fluid passageway for delivery to a patient and prevent the fluid from flowing through the fluid passageway for delivery to the patient.

83. A method, comprising:
causing fluid to flow through a fluid passageway of a sensor assembly for an infusion device, the sensor assembly comprising:
a body;
a pressure diaphragm coupled to the body, the pressure diaphragm comprising a diaphragm interface; and
a sensor comprising a sensor interface coupled to the diaphragm interface in an interlocked connection, the interlocked connection configured to transfer a load from the pressure diaphragm to the sensor;
wherein the fluid passageway is formed between the body and the pressure diaphragm;
determining, based on the load, a fluid pressure of the fluid;
detecting, based on a comparison of the fluid pressure to a threshold pressure, an occlusion in the fluid passageway; and
preventing, based on the detecting, delivery of the fluid to a patient.
